(19) United States
(12) Patent Application Publication
(10) Pub. No.: US 2019/0298038 A1
(43) Pub. Date: Oct. 3, 2019

(54) SKIN PATCH

(71) Applicant: Corey Miles, Toronto (CA)

(72) Inventor: Corey Miles, Toronto (CA)

(21) Appl. No.: 15/941,398

(22) Filed: Mar. 30, 2018

(51) Int. Cl. A45D 44/22; A61M 35/00; A61Q 19/08; A61K 8/04; A61K 8/02; A61K 8/73; A61K 8/65

(52) U.S. Cl. CPC A45D 44/22; A61M 35/00; A61Q 19/08; A61K 8/046; A61K 2800/28; A61K 8/044; A61K 8/731; A61K 8/65; A61K 8/0208

(57) ABSTRACT

Disclosed is a skin patch configured in a predefined shape and size that can fit generally over face, neck, and décolleté areas for anti-ageing, anti-wrinkle, and skin exfoliation applications. According to an embodiment, the skin patch includes a foam layer with a first surface and a second surface, a hydrocolloid layer with a top surface and an underside surface, the hydrocolloid layer is shaped and sized to conform to the shape and size of the foam layer, and wherein the top surface of the hydrocolloid layer is attached to the second surface of the foam layer, and a peel off layer shaped and sized to the shape and size of the hydrocolloid layer, wherein the peel off layer is removably attached to the underside surface of the hydrocolloid layer.

### TECHNICAL FIELD OF THE INVENTION

The present application generally relates to the field of cosmetics or skincare. Particularly, the application provides a skin patch provided for anti-ageing or anti-wrinkle or skin exfoliation applications especially for face, neck, and décolleté areas.

### BACKGROUND

Personal care has been one of the central aspects of human life. In general, people focus particularly on the face, along with other body parts, to maintain the appearance, vigor, and freshness of wrinkle-free facial skin. There has been a considerable industrial effort to develop various cosmetic solutions for facial treatments to attain such desired facial skin, which may include but not limited to facial patches, cosmetic compositions or solutions in the form of anti-aging or anti-wrinkle creams, surgical procedures, etc.

Aging of the face is known to result in reduced elasticity of the skin leaving wrinkles on the skin. It is known that a person can have some control over the acceleration of the wrinkle effect by maintaining good nutrition and exercise habits. Improvements can also be gained by application of various novel cosmetic skin care products and even cosmetic face lift surgeries, but those may not be cost effective solutions for all categories of people out there.

Apart from the natural process of aging that causes wrinkles, skin wrinkles may also be affected by sun damage, hormonal imbalance, muscle use, and gravity. Researchers have proved that wrinkles can also be greatly affected by sleep habits of people and is a growing concern in the world. A study published in the Aesthetic Surgery Journal (Volume 36, Issue 8, 1 Sep. 2016, Pages 931-940) indicates that compression, tension, and shear forces applied to the face during sleep can cause facial distortion when people sleep on their sides and stomach, leading to the development of sleep wrinkles over time. As can be seen in FIG. 1-2, during side or stomach sleeping, facial tissue is subject to shear, compression, and tensile, mechanical forces. The skin is stretched and pulled in all directions with changes in sleep position forcing the skin to undergo stress and hence create wrinkles over the time. Referring to FIG. 1-2, when the user lies down and\or sleeps on one's side throughout the night, the facial skin folds and falls back around different areas of face (as shown highlighted in FIG. 2), such as the under eye area. This results in wrinkles that are particularly noticeable by the user in the morning after waking up from sleep. The depth of the wrinkles usually reduces during the day while in the upright position as gravity causes the skin to be pulled down reducing the wrinkles. However, over time, these wrinkles can become permanent and the depth of the wrinkles can increase, as shown highlighted in FIG. 2.

Many manufacturers have proposed several solutions such as, DermaGen Anti-Wrinkle Patches. According to the manufacturer, the DermaGen patches are designed to minimize and flatten raised scar areas, while also reducing wrinkles.

There are also several anti wrinkle creams or compositions available in the art that can be applied over the skin directly and claim to reduce the appearance of fine lines and wrinkles on face. Such anti wrinkle creams or compositions usually contain active ingredients such as antioxidants and vitamins for reducing the wrinkles.

Furthermore several inventors in the past have envisioned different types of skin/facial patches for reducing wrinkles on the skin, for example, U.S. Pat. No. 9,854,896 discloses a wrinkle reducing pad. The US patent discloses a wrinkle pad with an adhesive layer that's covered by a protective layer that needs to be peeled off before applying the patch over the skin. The patch as disclosed in the patent includes a tension strap on the outer surface of the adhesive pad, which is removable from one end and is fixedly secured at the other end. When the user desires to use the wrinkle reducing pad on facial skin, the user needs to pull away the tension strap from the adhesive pad and attach the adhesive layer of the pad over the skin and apply pressure from the top of the pad and then again re-place the pulled away strap over the pad and leave it for some time. When the user removes the adhesive pad from the skin, the user needs to pull away the tension strap again and then pull the adhesive layer attached over the skin to remove the adhesive pad for reuse at a later time. Although such patches may help in reducing the wrinkles, there is always a risk that the adhesive pad may itself get folded along with the skin while the user is sleeping and may fail to prevent the skin from getting folded.

Thus, in the light of the above mentioned background discussion, it is evident that, there is a need for a face patch that may be easier to use and may hold the skin more tightly and prevent it from getting folded while the user sleeps with the patch over skin. Further there is a need to provide a face patch or a skin patch that may be impregnated with antioxidants and other anti-aging ingredients, which may be used for anti-ageing, anti-wrinkle, and skin exfoliation applications.

### SUMMARY

Before the present systems and methods, enablements are described, it is to be understood that this application is not limited to the particular systems, and methodologies described, as there can be multiple possible embodiments which are not expressly illustrated in the present disclosures. It is also to be understood that the terminology used in the description is for the purpose of describing the particular versions or embodiments only, and is not intended to limit the scope of the present application.

It is an object of the present invention to provide a skin patch, especially for face, neck, and décolleté areas.

According to another objective of the present invention, a face or skin patch is provided for anti-ageing, anti-wrinkle, and skin exfoliation applications.

According to an embodiment of the present invention, a skin patch is configured in a predefined shape and size, the skin patch fits generally over face, neck, and décolleté areas for anti-ageing, anti-wrinkle, and skin exfoliation applications. The skin patch comprises of a foam layer with a first surface and a second surface; a hydrocolloid layer with a top surface and an underside surface, the hydrocolloid layer is shaped and sized to conform to the shape and size of the foam layer, and wherein the top surface of the hydrocolloid layer is attached to the second surface of the foam layer; and a peel off layer shaped and sized to the shape and size of the hydrocolloid layer, wherein the peel off layer is removably attached to the underside surface of the hydrocolloid layer.

According to another embodiment, a skin patch configured in a predefined shape and size is provided to fit generally over face, neck, and décolleté areas for anti-ageing, anti-wrinkle, and skin exfoliation applications. The skin patch comprising of a foam layer with a first surface and a second surface; a hydrocolloid layer with a top surface and an underside surface, the hydrocolloid layer is shaped to conform to the shape of the foam layer, whereas sized slightly larger than the size of the foam layer, wherein the top surface of the hydrocolloid layer is attached to the second surface of the foam layer; and a peel off layer shaped and sized to the shape and size of the hydrocolloid layer, wherein the peel off layer is removably attached to the underside surface of the hydrocolloid layer.

Embodiment of the present invention discloses a skin patch that may be provided in different shapes and sizes to cover face, neck, and décolleté areas. For example, the skin patch may be provided in two halves for upper face part and lower face part to cover a human face. The skin patch may be provided in a size and shape that fit generally in the area directly below the user's eye, and also other face areas such as forehead, mouth, cheek. Furthermore, the skin patch may be provided in many different generic shapes for targeted patching or applying to specific areas of the human face, neck, and décolleté areas.

### BRIEF DESCRIPTION OF THE DRAWINGS

The foregoing summary, as well as the following detailed description of the preferred embodiments, is better understood when read in conjunction with the appended drawings. Example embodiments are shown in the drawings, however, the application is not limited to the specific system and method disclosed in the drawings.

FIG. 1-2 shows example illustrations to demonstrate how human sleep habits can lead to sleep wrinkles, especially when the users sleep on their side and/or stomach.

FIG. 3 shows front views of exemplary skin patches configured in a predefined shape and size to fit generally over face, neck, and décolleté areas for anti-ageing, anti-wrinkle, and skin exfoliation applications.

FIG. 4 shows a skin patch configured in a predefined shape and size, according to an embodiment of the present invention.

FIG. 5 shows a skin patch configured in a predefined shape and size, according to another embodiment of the present invention.

FIG. 6 shows representation of a human face, in particular a woman's face with exemplary skin patch applied to the face.

### DETAILED DESCRIPTION

In the following detailed description of the embodiments of the present subject matter, references are made to the accompanying drawings that form a part hereof, and in which are shown by way of illustration, specific embodiments in which the present subject matter may be practiced. These embodiments are described in sufficient detail to enable those skilled in the art to practice the present subject matter, and it is to be understood that other embodiments may be utilized and that changes may be made without departing from the scope of the present subject matter. The following detailed description is, therefore, not to be taken in a limiting sense, and the scope of the present subject matter is defined by the appended claims.

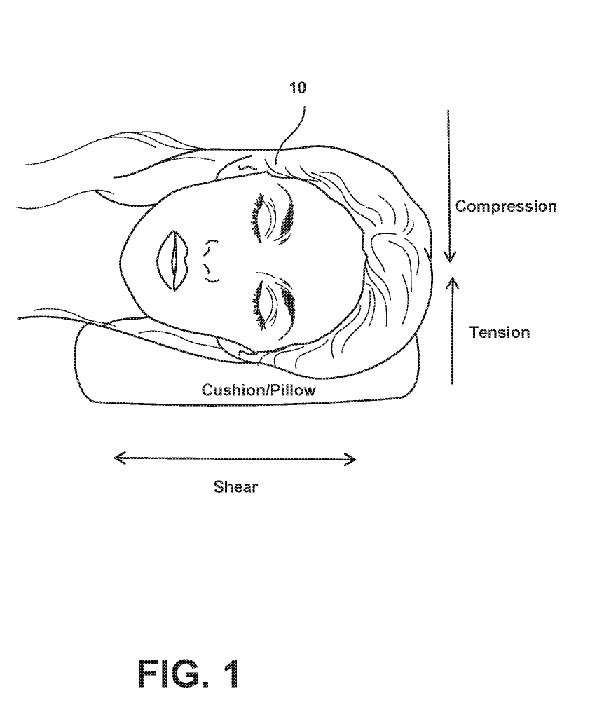

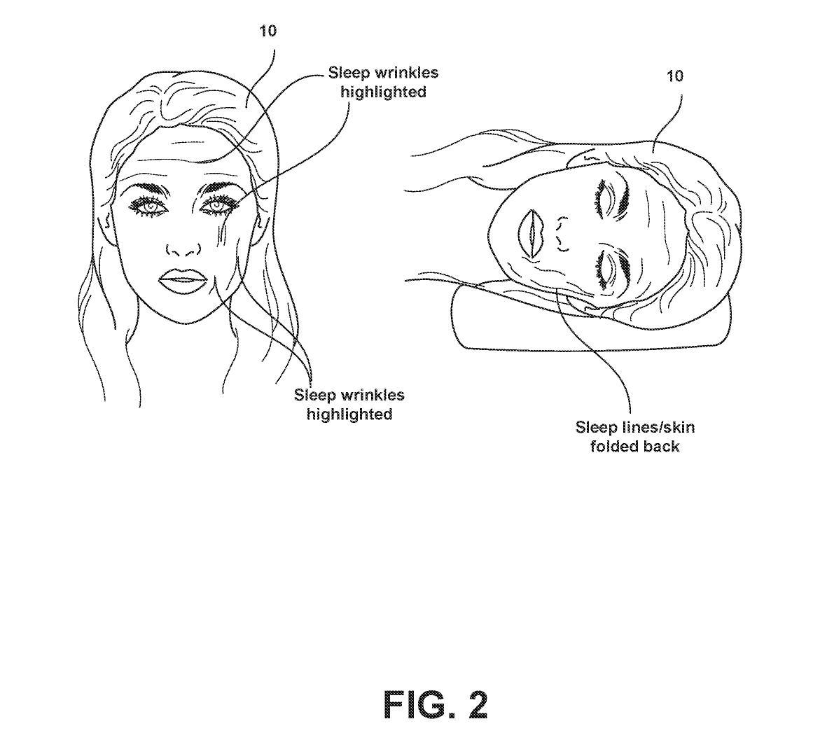

The accompanying FIG. 1-2 describes the concept of sleep wrinkles in humans 10. From FIGS. 1-2 it is clear that sleep wrinkles are developed due to improper sleep positions. During side sleeping or stomach sleeping, facial tissues, neck tissues, and décolleté tissues are subject to shear, compression, and tensile forces as shown in FIG. 1. The skin is generally stretched and pulled in all directions with changes in sleep positions.

Initially, wrinkles caused by facial expression begin as several, small, superficial creases perpendicular to the axis of muscle contraction and parallel to the axis of skin compression. As skin loses its elasticity and stiffens, they progress to fewer, deeper sleep lines which become permanent with repetition of the same sleep habit, as shown highlighted in FIG. 2.

Also, it is known that wrinkles can develop in different areas of face, neck, and/or décolleté including underneath the eyes. Keeping this in mind, the invention is directed towards a skin patch configured in predefined shapes and sizes that may fit generally over face, neck, and décolleté areas.

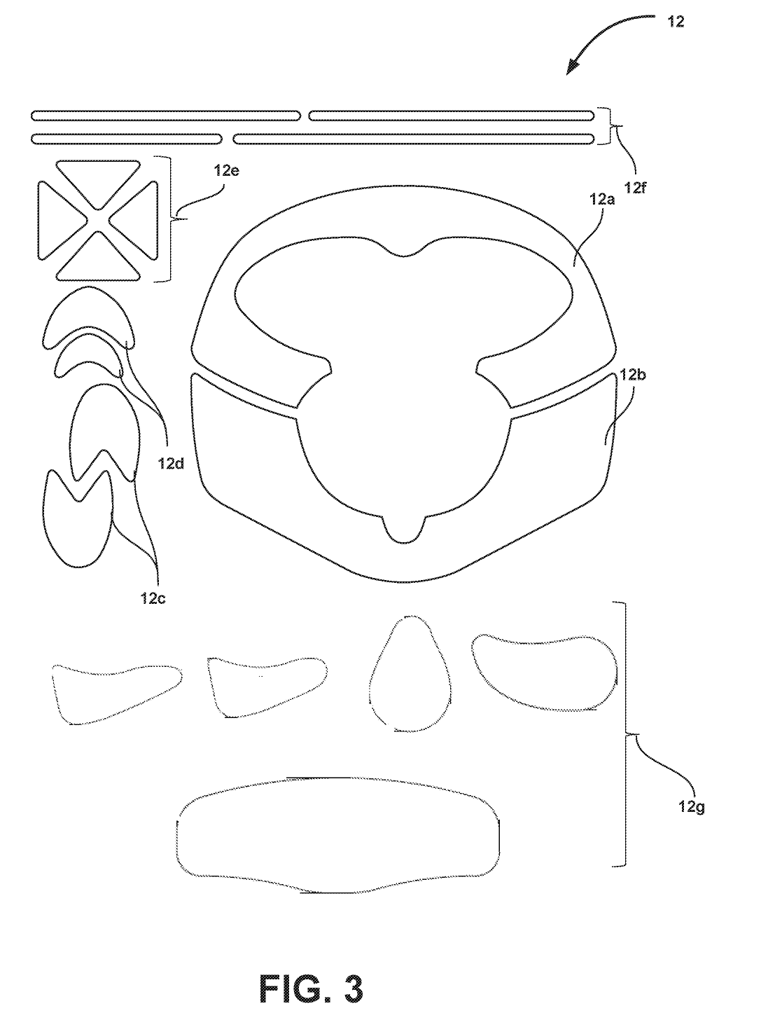

Referring to FIG. 3 that shows some exemplary configurations of skin patch of the proposed invention which can be suitably used over face, neck, and décolleté areas for anti-ageing, anti-wrinkle, and skin exfoliation applications. As seen, the skin patch 12 may comprise of two halves, an upper part 12a and a lower part 12b corresponding to an upper face part and a lower face part to cover a human face. The skin patch 12 may further comprise of a set of shapes that can be applied preferably near and under the eyes say 12c, 12d, 12g a plurality of generic shapes 12e, 12f for other targeted patching or applying to specific areas of the human face. The scope of invention should not be limited to the various exemplary shapes with varying sizes shown in the FIG. 3. There can be many other possible shapes with different sizes that can be derived based on users need.

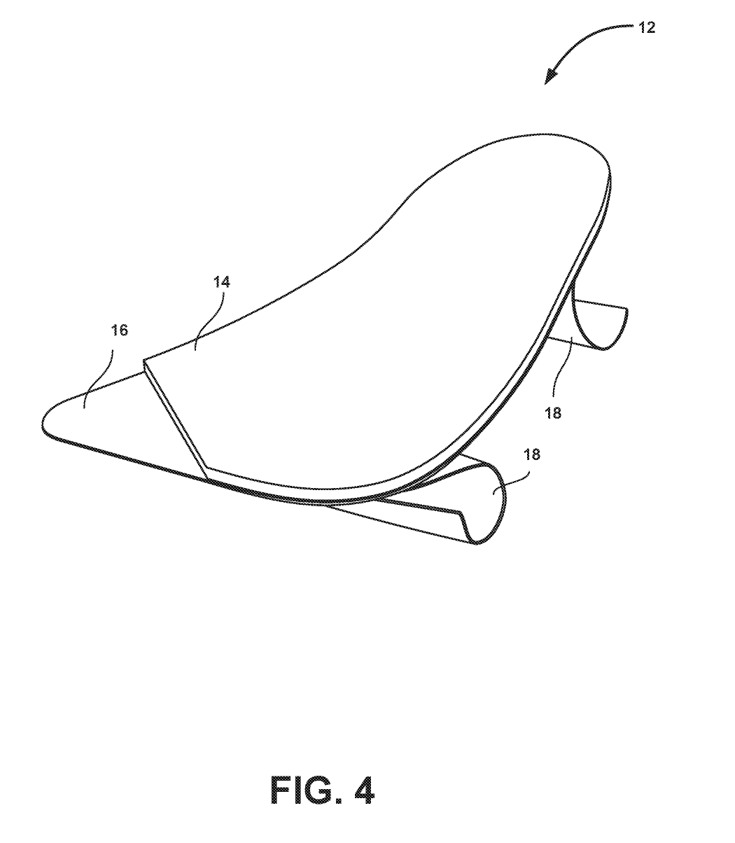

Referring to FIG. 4, which shows an exemplary skin patch 12 configured in a predefined shape and size, according to an embodiment. For the purpose of explanation, the skin patch 12 of specific shape and size that may be applied under the eye is considered, but it should be understood that any other shapes disclosed in FIG. 3 will embody the same features.

As seen in FIG. 4, the skin patch 12 includes a top foam layer 14. The foam layer 14 includes a first surface and a second surface. The foam layer 14 may be made up of a 1-2 mm medical-grade soft-foam, which may be medically-certified, closed-cell soft-touch foam. Preferably, the foam layer is about 1-2 mm thick.

The skin patch 12 further includes a thin layered base 16 such as hydrocolloid layer or other similar compositions. The other similar compositions may be selected from a group comprising but not limited to silicone, hydrogel, adhesive, tape, kraft paper, collagen and transparent film dressing. The hydrocolloid layer 16 is tacky on its underside surface, and may work for the single-use scenario, while still maintaining good adhesion through the night while in use. Although as explained herein, the patch 12 is preferably used one time, however, it should be understood that the skin patch 12 can be used as many times until the skin patch 12 loses its stickiness or wears off. The hydrocolloid layer 16 is shaped and sized to conform to the shape and size of the foam layer 14, meaning the size and shape of the foam layer 14 and the hydrocolloid layer 16 is identical. According to the embodiment, the top surface of the hydrocolloid layer 16 is preferably fixedly attached to the second surface of the foam layer 14. The foam layer 14 may be permanently bonded at the time of manufacturing using its own inbuilt medical-adhesive layer directly to the hydrocolloid layer 16. Basically both the hydrocolloid layer 16 and the foam layer 14 have viscidity which help them in bond formation between them. The hydrocolloid layer 16 is moisture retentive and comprises of an adhesive rubber matrix that facilitates attachment of the top surface of the hydrocolloid layer 16 to the second surface of the foam layer 14. The adhesive rubber matrix preferably comprises of a gel-forming colloidal suspension formed of at least one of a carboxymethylcellulose, a hydroxymethylcellulose, gelatin, polymers and adhesives. The hydrocolloid layer 16 is about 0.3-1 mm thick.

As seen in FIG. 4, the skin patch 12 further includes a lower peel off layer 18 shaped and sized identical to the shape and size of the hydrocolloid layer 16. According to an embodiment, the peel off layer 18 may be in two pieces as shown in FIG. 4. According to another embodiment, the peel off layer 18 may be available as a single piece. The peel off layer 18 is removably attached to the underside adhesive surface of the hydrocolloid layer 16. The underside of the hydrocolloid layer 16 is sticky in nature due to the presence of a gel-forming colloidal suspension discussed above which facilitates attachment of the peel off layer 18 over the underside layer of the hydrocolloid layer 16. Just before the skin patch 12 is to be applied to the skin of a user at a desired location, two ends of the peel off layer 18 is lifted away from the hydrocolloid layer 16 as shown in the FIG. 4, and the peel off layer 18 is grasped, e.g., between a thumb and forefinger and completely pulled away from the hydrocolloid layer 16. Although it is shown that the peel off layer 18 is pulled away as two different parts according to the embodiment, it is equally possible to pull away the peel off layer 18 as a single layer if the peel off layer 18 is made as a single layer.

The hydrocolloid layer 16 permanently holds the skin in place and doesn't let the skin fold back that may give rise to sleep lines which transforms to wrinkles. The top foam layer 14 provides firm application over the skin and does not let the hydrocolloid layer 16 fold back along with the skin, hence producing better results.

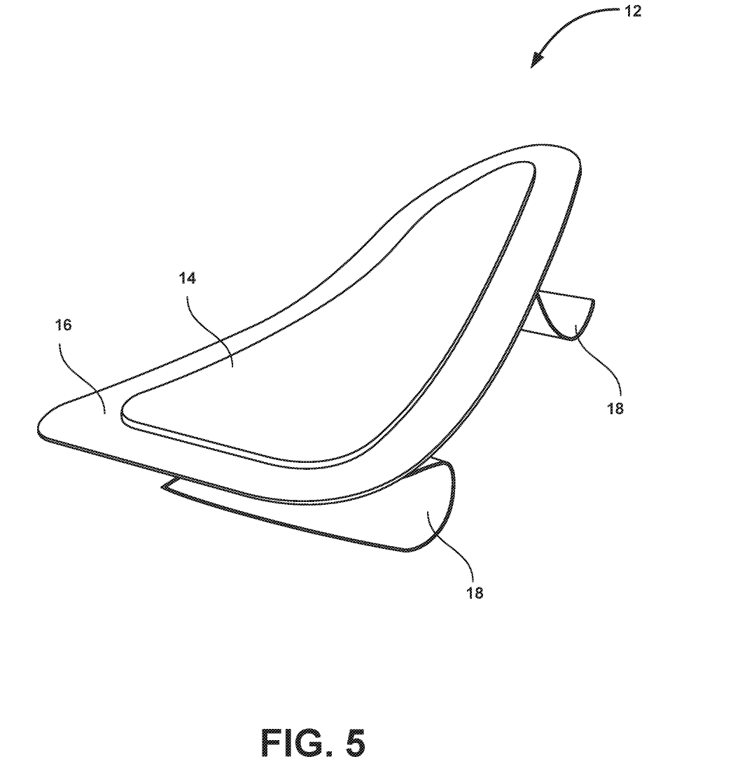

FIG. 5 shows alternative embodiment of the skin patch 12. The features, constituents forming each of the layers 14, 16, 18 of the skin patch 12 and functionalities remain identical as described above with respect to the FIG. 4. As shown, it is envisioned that the hydrocolloid layer 16 may be made slightly larger in size compared to the foam layer such that the foam layer 14 and the hydrocolloid layer 16 do not meet each other in their periphery unlike that shown in first embodiment in the FIG. 4.

The skin patch 12, in particular the underside surface of the hydrocolloid layer 16 of the proposed invention may be selectively impregnated with antioxidant and anti-aging ingredients that claim to reduce wrinkles. The antioxidant and anti-aging ingredients preferably include but are not limited to hyaluronic acid, ceramides, niacinamide, peptides, vitamin C, vitamin A, beta hydroxy acid, alpha hydroxy acid, CBD (cannabidiol). The skin patch 12 is generally selectively impregnated with antioxidants and anti-aging ingredients during the manufacturing process.

The skin patch 12 may be layered with a release liner 18 (as shown in FIG. 4), wherein the foam layer 14 and the hydrocolloid layer 16 may not be separated, even while removing the skin patch 12 after application, due to the ultra-strong medical-grade adhesion layer present between the foam layer 14 and the hydrocolloid layer 16.

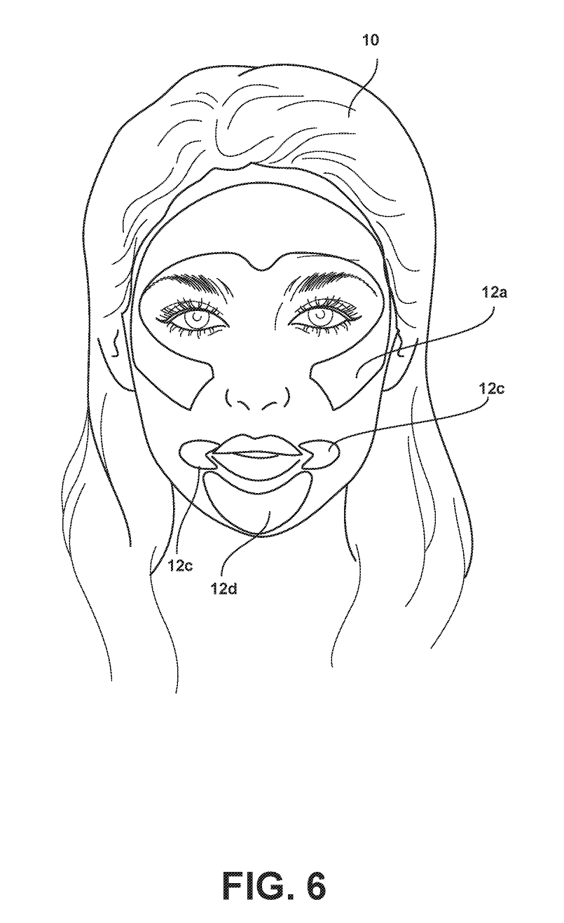

FIG. 6 shows representation of a human face, in particular a woman's face 10 with exemplary skin patches 12a, 12c, and 12d applied to the face. Just before the skin patch 12 (any of 12a, 12c, or 12d) is applied to the skin of the user at a desired location, the peel off layer 18 is lifted away from the hydrocolloid layer 16. The underside layer of the hydrocolloid layer 16 is then adhered to the skin and a slight pressure is applied over the foam layer 14 so that the hydrocolloid layer 16 gets firmly fixed over the skin in order to hold the skin and not to let the skin get folded which may result in the development of sleep lines (which transforms into sleep wrinkles with time), while the user sleeps on their side or sleeps on their stomach.

Although the skin patch of the present invention is mainly developed for face, neck and décolleté areas (used as a face patch) for prevention of sleep wrinkles that gradually develop due to improper sleep habits, it may also be used for application to other regions of the body as well. In addition to being used as the face patch, the proposed skin patch may also help exfoliate dead skin leaving behind smoother, more youthful-looking skin.

The preceding description has been presented with reference to various embodiments. People skilled in the art and technology to which this application pertains will appreciate that alterations and changes in the described structures and methods of operation can be practiced without meaningfully departing from the principle, spirit and scope of the presented disclosure.

What is claimed is:

1. A skin patch 12 configured in a predefined shape and size, the skin patch 12 fits generally over face, neck, and décolleté areas for anti-ageing, anti-wrinkle, and skin exfoliation applications, the skin patch 12 comprises of:
a foam layer 14 with a first surface and a second surface;
a hydrocolloid layer 16 with a top surface and an underside surface, the hydrocolloid layer 16 is shaped and sized to conform to the shape and size of the foam layer 14, and wherein the top surface of the hydrocolloid layer 16 is attached to the second surface of the foam layer 14; and
a peel off layer 18 shaped and sized to the shape and size of the hydrocolloid layer 16, wherein the peel off layer 18 is removably attached to the underside surface of the hydrocolloid layer 16.

2. The skin patch 12 of claim 1, wherein the foam layer 14 is about 1-2 mm thick.

3. The skin patch 12 of claim 1, wherein the hydrocolloid layer 16 is about 0.3-1 mm thick.

4. The skin patch 12 of claim 1, wherein the underside surface of the hydrocolloid layer 16 is sticky in nature to removably attach to the peel off layer 18.

5. The skin patch 12 of claim 1, wherein the hydrocolloid layer 16 is moisture retentive and comprises of an adhesive rubber matrix that enables attachment of the top surface of the hydrocolloid layer 16 to the second surface of the foam layer 14.

6. The skin patch 12 of claim 5, wherein the adhesive rubber matrix comprises of a gel-forming colloidal suspension formed of at least one of a carboxymethylcellulose, a hydroxymethylcellulose, gelatin, polymers and adhesives.

7. The skin patch 12 of claim 1, wherein the underside surface of the hydrocolloid layer 16 is selectively impregnated with antioxidant and anti-aging ingredients.

8. The skin patch 12 of claim 1, wherein the foam layer 14 is made up of medical-grade closed-cell soft-touch foam.

9. A skin patch 12 configured in a predefined shape and size, the skin patch 12 fits generally over face, neck, and décolleté areas for anti-ageing, anti-wrinkle, and skin exfoliation applications, the skin patch 12 comprising:
a foam layer 14 with a first surface and a second surface;
a hydrocolloid layer 16 with a top surface and an underside surface, the hydrocolloid layer 16 is shaped to conform to the shape of the foam layer 14, whereas sized slightly larger than the size of the foam layer 14, wherein the top surface of the hydrocolloid layer 16 is attached to the second surface of the foam layer 14; and
a peel off layer 18 shaped and sized to the shape and size of the hydrocolloid layer 16, wherein the peel off layer 18 is removably attached to the underside surface of the hydrocolloid layer 16.

10. The skin patch 12 of claim 9, wherein the foam layer 14 is about 1-2 mm thick.

11. The skin patch 12 of claim 9, wherein the hydrocolloid layer 16 is about 0.3-1 mm thick.

12. The skin patch 12 of claim 9, wherein the underside surface of the hydrocolloid layer 16 is sticky in nature to removably attach to the peel off layer 18.

13. The skin patch 12 of claim 9, wherein the hydrocolloid layer 16 is moisture retentive and comprises of an adhesive rubber matrix that enables attachment of the top surface of the hydrocolloid layer 16 to the second surface of the foam layer 14.

14. The skin patch 12 of claim 13, wherein the adhesive rubber matrix comprises of a gel-forming colloidal suspension formed of at least one of a carboxymethylcellulose, a hydroxymethylcellulose, gelatin, polymers and adhesives.

15. The skin patch 12 of claim 9, wherein the underside surface of the hydrocolloid layer 16 is selectively impregnated with antioxidant and anti-aging ingredients.

16. The skin patch 12 of claim 9, wherein the foam layer 14 is made up of medical-grade closed-cell soft-touch foam.
